(19) United States
(12) Patent Application Publication
(10) Pub. No.: US 2023/0004862 A1
(43) Pub. Date: Jan. 5, 2023

(54) METHOD FOR TRAINING RANKING LEARNING MODEL, RANKING METHOD, DEVICE AND MEDIUM

(71) Applicant: BEIJING BAIDU NETCOM SCIENCE TECHNOLOGY CO., LTD., Beijing (CN)

(72) Inventors: Yingfei XIANG, Beijing (CN); Hongyu LUO, Beijing (CN); Xiaomin FANG, Beijing (CN); Fan WANG, Beijing (CN)

(73) Assignee: BEIJING BAIDU NETCOM SCIENCE TECHNOLOGY CO., LTD., Beijing (CN)

(21) Appl. No.: 17/537,575

(22) Filed: Nov. 30, 2021

(30) Foreign Application Priority Data
Jun. 30, 2021 (CN) 202110739297.3

(51) Int. Cl. G06N 20/00

(52) U.S. Cl. CPC G06N 20/00

(57) ABSTRACT

The technical solution relates to the field of artificial intelligence technologies, such as machine learning technologies, natural language processing technologies, or the like. A plurality of training samples are collected, each of the plurality of training samples includes information of a known training target protein, information of two training drugs, and a real difference between affinities of the two training drugs for the known training target. The ranking learning model is trained with the plurality of training samples, such that the ranking learning model learns a capability of predicting a magnitude relationship between the affinities of the two training drugs for the known training target protein in each of the plurality of training samples.

### CROSS-REFERENCE TO RELATED APPLICATIONS

The present disclosure claims priority and benefit of Chinese Patent Application No. 202110739297.3, filed on Jun. 30, 2021, entitled “METHOD AND APPARATUS FOR RANKING LEARNING MODEL, RANKING METHOD AND APPARATUS, DEVICE AND MEDIUM”. The disclosure of the above application is incorporated herein by reference in its entirety.

### TECHNICAL FIELD

The present disclosure relates to the field of computer technologies, and particularly to the field of artificial intelligence technologies, such as machine learning technologies, natural language processing technologies, or the like, and more particularly to a method for training a ranking learning model, a ranking method, a device and a medium.

### BACKGROUND

A drug target interaction (DTI), which represents an affinity between a target protein and a drug compound, is a quite important part in the field of drug development. The DTI may assist a drug developer in understanding a mechanism of a disease to speed up a drug design process.

In the traditional biological field, a method for detecting the DTI in a laboratory using a wet experiment is quite expensive and time-consuming. Nowadays, with maturity of a deep learning algorithm based on artificial intelligence (AI), more and more DTI tasks are implemented using network models, such as a graph neural network (GNN), a convolutional neural network (CNN), or the like.

### SUMMARY

The present disclosure provides a method for training a ranking learning model, a ranking method, a device and a medium.

According to an aspect of the present disclosure, there is provided a method for training a ranking learning model, including collecting a plurality of training samples, each of the plurality of training samples including information of a known training target protein, information of two training drugs, and a real difference between affinities of the two training drugs for the known training target protein; and training the ranking learning model with the plurality of training samples, such that the ranking learning model learns a capability of predicting a magnitude relationship between the affinities of the two training drugs for the known training target protein in each of the plurality of training samples.

According to another aspect of the present disclosure, there is provided a drug ranking method, including acquiring information of an objective target and information of a plurality of candidate drugs; and ranking the plurality of candidate drugs according to magnitude of their respective affinities for the objective target by a ranking model based on the information of the objective target and the information of the plurality of candidate drugs, the ranking model sharing parameters of a pre-trained ranking learning model, and the ranking learning model being configured to learn a magnitude relationship between affinities of any two drugs for a same target protein.

According to another aspect of the present disclosure, there is provided an electronic device, including at least one processor; and a memory connected with the at least one processor communicatively; the memory stores instructions executable by the at least one processor to enable the at least one processor to carry out the method of the aspect as described above and any possible implementation.

According to still another aspect of the present disclosure, there is provided a non-transitory computer readable storage medium including computer instructions, which, when executed by a computer, cause the computer to carry out the method of the aspect as described above and any possible implementation.

It should be understood that the statements in this section are not intended to identify key or critical features of the embodiments of the present disclosure, nor limit the scope of the present disclosure. Other features of the present disclosure will become apparent from the following description.

### BRIEF DESCRIPTION OF THE DRAWINGS

The drawings are used for better understanding the present solution and do not constitute a limitation of the present disclosure. In the drawings,

FIG. 1 is a schematic diagram according to a first embodiment of the present disclosure;

FIG. 2 is a schematic diagram according to a second embodiment of the present disclosure;

FIG. 3 is a schematic diagram according to a third embodiment of the present disclosure;

FIG. 4 is a schematic diagram according to a fourth embodiment of the present disclosure;

FIG. 5 is a schematic diagram according to a fifth embodiment of the present disclosure;

FIG. 6 is a schematic diagram according to a sixth embodiment of the present disclosure;

FIG. 7 is a schematic diagram according to a seventh embodiment of the present disclosure; and

FIG. 8 shows a schematic block diagram of an exemplary electronic device 800 which may be configured to implement the embodiments of the present disclosure.

### DETAILED DESCRIPTION

The following part will illustrate exemplary embodiments of the present disclosure with reference to the drawings, including various details of the embodiments of the present disclosure for a better understanding. The embodiments should be regarded only as exemplary ones. Therefore, those skilled in the art should appreciate that various changes or modifications can be made with respect to the embodiments described herein without departing from the scope and spirit of the present disclosure. Similarly, for clarity and conciseness, the descriptions of the known functions and structures are omitted in the descriptions below.

Apparently, the embodiments to be described are merely some rather than all of the embodiments of the present disclosure. All other embodiments obtained by a person of ordinary skill in the art based on the embodiments of the present disclosure without creative efforts shall fall within the protection scope of the present disclosure.

It should be noted that a terminal device in the embodiments of the present disclosure may include, but is not limited to, a mobile phone, a personal digital assistant (PDA), a wireless handheld device, a tablet computer, and other smart devices; a display device may include, but not limited to, a personal computer, a television, and other devices with a display function.

In addition, the term “and/or” only describes an association relationship between associated objects, and indicates that three relationships may exist. For example, A and/or B may indicate three cases: only A exists; both A and B exist; and only B exists. In addition, in this specification, the symbol “/” generally indicates that associated objects have a relationship of “or”.

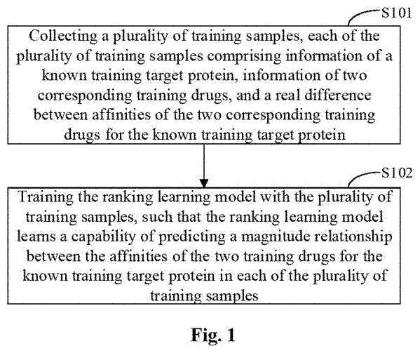

FIG. 1 is a schematic diagram according to a first embodiment of the present disclosure; as shown in FIG. 1, this embodiment provides a method for training a ranking learning model. As shown in FIG. 1, the method for training a ranking learning model according to this embodiment may include the following steps:

S101: collecting a plurality of training samples, each of the plurality of training samples including information of a known training target protein, information of two corresponding training drugs, and a real difference between affinities of the two corresponding training drugs for the known training target; and

S102: training the ranking learning model with the plurality of training samples, such that the ranking learning model learns a capability of predicting a magnitude relationship between the affinities of the two training drugs for the known training target protein in each of the plurality of training samples.

An apparatus for training a ranking learning model serves as the subject for executing the method for training a ranking learning model according to this embodiment, and an electronic entity or a software-integrated application may serve as the executing subject of the apparatus for training a ranking learning model. The apparatus for training a ranking learning model according to this embodiment is configured to train a ranking learning model.

The ranking learning model in this embodiment is configured to learn to predict the magnitude relationship between the affinities of the two training drugs for the known training target protein, and then rank a plurality of training drugs according to the magnitude of affinities of the plurality of training drugs for the known training target protein based on the magnitude relationship between the affinities of every two training drugs for the known training target protein.

A plurality of training samples are collected in this embodiment, and each training sample includes the information of the two training drugs; for example, the information of the training drug may be identified using a simplified molecular input line entry specification (SMILES) sequence of the training drug, or may be other unique identification information of the training drug. The information of the known training target protein may be identified using a FASTA sequence of the known training target protein, or may be other unique identifying information of the known training target protein.

It should be noted that since each training sample in this embodiment is used for training the ranking learning model to learn the capability of predicting the magnitude relationship between the affinities of the two training drugs for the known training target protein in each training sample, in a supervised training operation, each training sample in this embodiment further includes the real difference between the affinities of the two training drugs for the known training target protein; that is, the real difference between the affinities may identify the magnitude relationship between the affinities of the two training drugs for the known training target protein. Based on this, optionally, in an actual operation, the real difference between the affinities may be a specific difference value, or may only identify a direction of the real difference between the affinities. For example, for two training drugs A and B, if the affinity a of the training drug A for a known training target protein 1 is greater than the affinity b of the training drug B for the known training target protein 1 (i.e., a−b>0), the corresponding real difference between the affinities may be identified to be 1, and if the affinity a of the training drug A for the known training target protein 1 is less than the affinity b of the training drug B for the known training target protein 1 (i.e., a−b<0), the corresponding real difference between the affinities may be identified to be 0.

Then, based on the information of the two training drugs and the real difference between the affinities of the two corresponding training drugs for a known training target in each of the plurality of training samples, the ranking learning model may be subjected to a supervised training operation to learn the real difference between the affinities of the two training drugs for the known training target which are identified in each training sample, and the ranking learning model is continuously trained using the plurality of training samples, so as to learn the capability of predicting the magnitude relationship between the affinities of the two training drugs for the known training target protein in each training sample.

In this embodiment, the number of collected training samples may be huge, for example, hundreds of thousands or even millions. The greater the number of the training samples, the higher the accuracy of the trained ranking learning model.

In the method for training a ranking learning model according to this embodiment, the ranking learning model is trained using each training sample including the information of the known training target protein, the information of the two corresponding training drugs, and the real difference between the affinities of the two corresponding training drugs for the known training target, such that the ranking learning model may learn the capability of predicting the magnitude relationship between the affinities of the two training drugs for the known training target protein in each training sample.

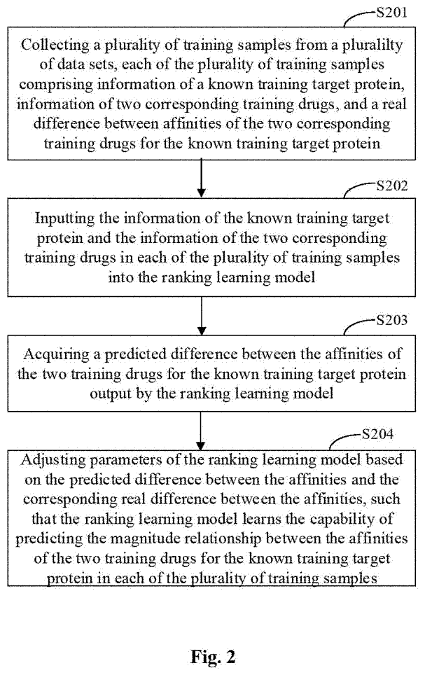

FIG. 2 is a schematic diagram according to a second embodiment of the present disclosure; as shown in FIG. 2, the technical solution of the method for training a ranking learning model according to this embodiment of the present disclosure is further described in more detail based on the technical solution of the above-mentioned embodiment shown in FIG. 1. As shown in FIG. 2, the method for training a ranking learning model according to this embodiment may include the following steps:

5201: collecting a plurality of training samples from a plurality of data sets, each training sample including information of a known training target protein, information of two corresponding training drugs, and a real difference between affinities of the two corresponding training drugs for the known training target.

Optionally, in this embodiment, the affinities of the training drugs for the known training target in different data sets may be characterized by different indexes. For example, the indexes of the affinities in some data sets are identified by IC50, the indexes of the affinities in some data sets are identified by Kd, and the indexes of the affinities in some data sets are identified by Ki. No matter which affinity index is adopted in the data set, the training sample in this embodiment is only required to identify the direction of the real difference between the affinities of the two training drugs for the known training target.

For example, in the schematic diagram of training sample construction shown in FIG. 3, a training set formed by a plurality of collected training samples may include m training target proteins, which may be identified as t⁽¹⁾, . . . , t^{(m)} respectively. For each training target protein, n training drugs and the affinity of respective training drugs for the training target protein may be collected first; for example, for the training target protein t⁽¹⁾, the collected training drugs may be denoted as {(d₁⁽¹⁾, S₁⁽¹⁾), (d₂⁽¹⁾, S₂⁽¹⁾), . . . , (dₙ⁽¹⁾, Sₙ⁽¹⁾)}; for the training target protein t^{(m)}, the collected training drugs may be denoted as {(d₁^{(m)}, S₁^{(m)}), (d₂^{(m)}, S₂^{(m)}), . . . , (dₙ^{(m)}, Sₙ^{(m)})}. For a single target protein, all corresponding drugs d may form a pairwise relationship, and for each pair of drugs (dᵢ^{(m)}, dⱼ^{(m)}, the difference between corresponding affinity scores may be written as s(Sᵢ^{(m)}, Sⱼ^{(m)}). As shown in FIG. 3, for the training target protein t⁽¹⁾, any training sample may be denoted as f(t⁽¹⁾, dᵢ⁽¹⁾, dⱼ⁽¹⁾), s(Sᵢ⁽¹⁾, Sⱼ⁽¹⁾); or the training target protein t⁽²⁾, any training sample may be denoted as f(t⁽²⁾, dᵢ⁽²⁾, dⱼ⁽²⁾), s(Sᵢ⁽²⁾, Sⱼ⁽²⁾), and for the training target protein t^{(m)}, any training sample may be denoted as f(t^{(m)}, dᵢ^{(m)}, dⱼ^{(m)}), s(Sᵢ^{(m)}, Sⱼ^{(m)}).

The training drugs and the training target proteins may be from a plurality of different data sets, and the affinities of the training drugs corresponding to different training target proteins may be identified using different affinity indexes, only if the difference between the affinities of the two training drugs for the training target protein in any training sample is able to be identified. Similarly, a value of the difference between the affinities may not be identified, but only the direction of the difference is identified; that is, only the magnitude relationship is identified.

The ranking learning model in this embodiment may employ a neural network model, such as a multi-layer perceptron (MLP), a convolutional neural network (CNN), a Transformer, or the like, or may employ any other neural network structure capable of extracting and learning target protein or drug molecule characterization. The ranking learning model in this embodiment has a double-tower structure.

202: inputting the information of the known training target protein and the information of the two corresponding training drugs in each of the plurality of training samples into the ranking learning model.

203: acquiring a predicted difference between the affinities of the two training drugs for the known training target protein output by the ranking learning model.

204: adjusting parameters of the ranking learning model based on the predicted difference between the affinities and the corresponding real difference between the affinities, such that the ranking learning model learns the capability of predicting the magnitude relationship between the affinities of the two training drugs for the known training target protein in each of the plurality of training samples.

For example, the step may include the following steps during implementation:

(a) constructing a loss function based on the predicted difference between the affinities and the corresponding real difference between the affinities;

(b) detecting whether the loss function converges; if the loss function converges, executing step (d);

(c) if the loss function does not converge, adjusting the parameters of the ranking learning model, such that the loss function tends to converge; returning to step 202, selecting the next training sample, and continuing the training operation; and

(d) detecting whether a training termination condition is met, if yes, stopping the training operation, determining the parameters of the ranking learning model at this point, and ending the step; if no, returning to step 202, selecting the next training sample, and continuing the training operation.

Optionally, as the training termination condition in this embodiment, whether the loss function converges all the time in a preset continuous threshold number of training operations is detected, and if yes, the training termination condition is determined to be met. The preset continuous threshold number may be set according to an actual scenario, and may be, for example, 80, 100, 150 or other numbers, which is not limited herein. Alternatively, a maximum training number threshold may be set, and when the number of the training operations reaches the maximum training number threshold, the training operation is finished. The above-mentioned training mode may effectively improve an effect of training the ranking learning model.

The ranking learning model in this embodiment has a double-tower structure and achieves a ranking learning effect. The parameters of the learned ranking learning model may be shared by a ranking model having a single-tower structure, such that the ranking model may rank a plurality of drugs corresponding to a same target protein according to their respective affinities.

In the method for training a ranking learning model according to this embodiment, a full advantage may be taken of different data sets and DTI data of different indexes, and the magnitude relationship between the affinities of different drugs for the same target protein is learned by designing a ranking learning algorithm, thus ranking the plurality of drugs according to the affinities with the same target protein. Since more attention is paid to the difference between the affinities of the two paired drugs for the target protein in the training operation of the ranking learning model in this embodiment, cross-data-set data of plural affinity indexes may be integrated together to train the model, thus effectively overcoming a limitation that a DTI data set is small in the model training operation, and effectively improving the effect of training the ranking learning model.

In the method for training a ranking learning model according to this embodiment, the precedence relationship between the affinities of different drugs for the same target protein may be acquired by designing the pairwise ranking learning algorithm, and accuracy of ranking the affinities of different drugs for the same target protein may be effectively improved compared with other existing methods; for example, a weighted consistency index (WeightedCI) and an average consistency index (Averaged) of a corresponding drug based on a certain target protein may be increased by about 0.03 and 0.05 respectively.

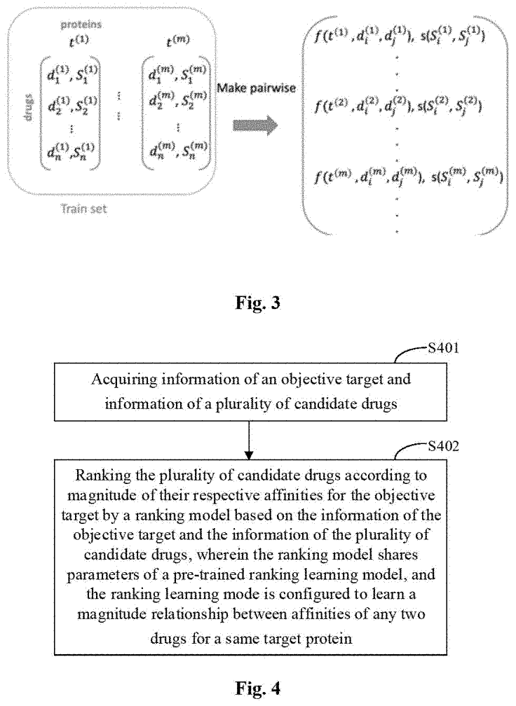

FIG. 4 is a schematic diagram according to a fourth embodiment of the present disclosure; as shown in FIG. 4, this embodiment provides a drug ranking method, which may include the following steps:

S401: acquiring information of an objective target and information of a plurality of candidate drugs; and

S402: ranking the plurality candidate drugs according to magnitude of their respective affinities for the objective target by a ranking model based on the information of the objective target and the information of the plurality of candidate drugs, herein the ranking model shares parameters of a pre-trained ranking learning model, and the ranking learning model is configured to learn a magnitude relationship between affinities of any two drugs for a same target protein.

A drug ranking apparatus serves as the subject for executing the drug ranking method according to this embodiment, and an electronic entity or a software-integrated application may serve as the executing subject of the drug ranking apparatus. The drug ranking operation in this embodiment is used for ranking the plurality of candidate drugs according to the magnitude of their affinities for the same target protein, and further may realize drug recommendation.

The ranking model in this embodiment has a single-tower structure, and the single-tower structure may be implemented by sharing the parameters of the ranking model trained in the embodiments shown in FIG. 1 or 2. The above-mentioned ranking learning model learns the magnitude relationship between the affinities of different drugs for the same target, thus ranking the plurality of drugs according to the magnitude of their affinities for the same target. For example, if the affinity of the drug A for the target 1 may be predicted to be greater than that of the drug B for the target 1, and meanwhile the affinity of the drug B for the target 1 may also be predicted to be greater than that of the drug C for the target 1, the drug A, the drug B, and the drug C may be ranked according to the magnitude of their affinities for the target 1, thus realizing drug recommendation.

Similarly, the information of the objective target in this embodiment may be identified using a SMILES sequence, and the information of the candidate drug may be identified using a FASTA sequence.

In use, the information of the objective target and the information of the plurality of candidate drugs are subjected to an embedding operation and then input into the ranking model, and the ranking model may predict and output, based on the input information, the ranking relationship for ranking the plurality of candidate drugs according to the magnitude of their affinities for the objective target protein. Subsequently, the drug with the highest affinity for the objective target protein may be obtained based on the ranking relationship, thus realizing the drug recommendation.

In the drug ranking method according to this embodiment, the ranking model shares the parameters of the pre-trained ranking learning model, the ranking learning model is configured to learn the magnitude relationship between the affinities of any two drugs for a same target protein, and the accuracy of ranking the drugs may be effectively improved using the ranking model, thus more effectively recommending the drug.

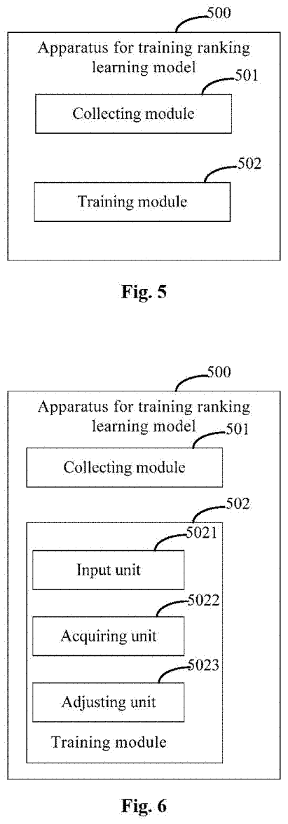

FIG. 5 is a schematic diagram according to a fifth embodiment of the present disclosure; as shown in FIG. 5, this embodiment provides an apparatus 500 for training a ranking learning model, including:

a collecting module 501 configured to collect a plurality of training samples, each of the plurality of training samples including information of a known training target protein, information of two corresponding training drugs, and a real difference between affinities of the two corresponding training drugs for the known training target; and

a training module 502 configured to train the ranking learning model with the plurality of training samples, such that the ranking learning model learns a capability of predicting a magnitude relationship between the affinities of the two training drugs for the known training target protein in each of the plurality of training samples.

The apparatus 500 for training a ranking learning model according to this embodiment has the same implementation as the above-mentioned relevant method embodiment by adopting the above-mentioned modules to implement the implementation principle and the technical effects of training the ranking learning model, and for details, reference may be made to the description of the above-mentioned relevant method embodiment, and details are not repeated herein.

FIG. 6 is a schematic diagram according to a sixth embodiment of the present disclosure; as shown in FIG. 6, the technical solution of the apparatus 500 for training a ranking learning model according to this embodiment of the present disclosure is further described in more detail based on the technical solution of the above-mentioned embodiment shown in FIG. 5.

As shown in FIG. 6, in the apparatus 500 for training a ranking learning model according to this embodiment, the training module 502 includes an input unit 5021 configured to input the information of the known training target protein and the information of the two corresponding training drugs in each of the plurality of training samples into the ranking learning model; an acquiring unit 5022 configured to acquire a predicted difference between the affinities of the two training drugs for the known training target protein output by the ranking learning model; and an adjusting unit 5023 configured to adjust parameters of the ranking learning model based on the predicted difference between the affinities and the corresponding real difference between the affinities, such that the ranking learning model learns the capability of predicting the magnitude relationship between the affinities of the two training drugs for the known training target protein in each of the plurality of training samples.

Further optionally, the adjusting unit 5023 is configured to construct a loss function based on the predicted difference between the affinities and the corresponding real difference between the affinities; detect whether the loss function converges; and if the loss function does not converge, adjust the parameters of the ranking learning model, such that the loss function tends to converge.

Further optionally, in the apparatus 500 for training a ranking learning model according to this embodiment, the collecting module 501 is configured to collect a plurality of training samples from a plurality of data sets.

The affinities of the training drugs for the known training targets in different data sets are characterized by different indexes.

The apparatus 500 for training a ranking learning model according to this embodiment has the same implementation as the above-mentioned relevant method embodiment by adopting the above-mentioned modules to implement the implementation principle and the technical effects of training the ranking learning model, and for details, reference may be made to the description of the above-mentioned relevant method embodiment, and details are not repeated herein.

FIG. 7 is a schematic diagram according to a seventh embodiment of the present disclosure; as shown in FIG. 7, this embodiment provides a drug ranking apparatus 700, including an acquiring module 701 configured to acquire information of an objective target and information of a plurality of candidate drugs; and a ranking module 702 configured to rank the plurality candidate drugs according to their respective affinities for the objective target by a ranking model based on the information of the objective target and the information of the plurality of candidate drugs, herein the ranking model shares parameters of a pre-trained ranking learning model, and the ranking learning model is configured to learn a magnitude relationship between affinities of any two drugs for a same target protein.

The drug ranking apparatus 700 according to this embodiment has the same implementation as the above-mentioned relevant method embodiment by adopting the above-mentioned modules to implement the implementation principle and the technical effects of ranking the drugs, and for details, reference may be made to the description of the above-mentioned relevant method embodiment, and details are not repeated herein.

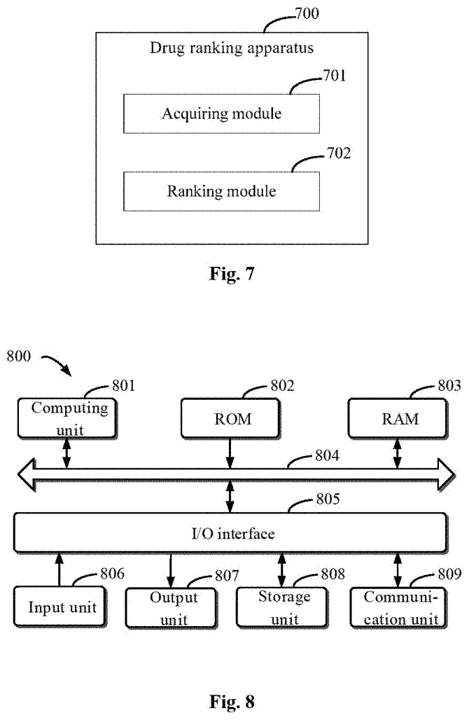

FIG. 8 shows a schematic block diagram of an exemplary electronic device 800 which may be configured to implement the embodiments of the present disclosure. The electronic device is intended to represent various forms of digital computers, such as laptop computers, desktop computers, workstations, servers, blade servers, mainframe computers, and other appropriate computers. The electronic device may also represent various forms of mobile devices, such as personal digital assistants, cellular telephones, smart phones, wearable devices, and other similar computing devices. The components shown herein, their connections and relationships, and their functions, are meant to be exemplary only, and are not meant to limit implementation of the present disclosure described and/or claimed herein.

As shown in FIG. 8, the electronic device 800 includes a computing unit 801 which may perform various appropriate actions and processing operations according to a computer program stored in a read only memory (ROM) 802 or a computer program loaded from a storage unit 808 into a random access memory (RAM) 803. Various programs and data necessary for the operation of the electronic device 800 may be also stored in the RAM 803. The computing unit 801, the ROM 802, and the RAM 803 are connected with one other through a bus 804. An input/output (I/O) interface 805 is also connected to the bus 804.

The plural components in the electronic device 800 are connected to the I/O interface 805, and include: an input unit 806, such as a keyboard, a mouse, or the like; an output unit 807, such as various types of displays, speakers, or the like; the storage unit 808, such as a magnetic disk, an optical disk, or the like; and a communication unit 809, such as a network card, a modem, a wireless communication transceiver, or the like. The communication unit 809 allows the electronic device 800 to exchange information/data with other devices through a computer network, such as the Internet, and/or various telecommunication networks.

The computing unit 801 may be a variety of general and/or special purpose processing components with processing and computing capabilities. Some examples of the computing unit 801 include, but are not limited to, a central processing unit (CPU), a graphic processing unit (GPU), various dedicated artificial intelligence (AI) computing chips, various computing units running machine learning model algorithms, a digital signal processor (DSP), and any suitable processor, controller, microcontroller, or the like. The computing unit 801 performs the methods and processing operations described above, such as the method for training a ranking learning model and the drug ranking method. For example, in some embodiments, the method for training a ranking learning model or the drug ranking method may be implemented as a computer software program tangibly contained in a machine readable medium, such as the storage unit 808. In some embodiments, part or all of the computer program may be loaded and/or installed into the electronic device 800 via the ROM 802 and/or the communication unit 809. When the computer program is loaded into the RAM 803 and executed by the computing unit 801, one or more steps of the method for training a ranking learning model or the drug ranking method described above may be performed. Alternatively, in other embodiments, the computing unit 801 may be configured to perform the method for training a ranking learning model or the drug ranking method by any other suitable means (for example, by means of firmware).

Various implementations of the systems and technologies described herein above may be implemented in digital electronic circuitry, integrated circuitry, field programmable gate arrays (FPGA), application specific integrated circuits (ASIC), application specific standard products (ASSP), systems on chips (SOC), complex programmable logic devices (CPLD), computer hardware, firmware, software, and/or combinations thereof. The systems and technologies may be implemented in one or more computer programs which are executable and/or interpretable on a programmable system including at least one programmable processor, and the programmable processor may be special or general, and may receive data and instructions from, and transmit data and instructions to, a storage system, at least one input device, and at least one output device.

Program codes for implementing the method according to the present disclosure may be written in any combination of one or more programming languages. These program codes may be provided to a processor or a controller of a general purpose computer, a special purpose computer, or other programmable data processing devices, such that the program code, when executed by the processor or the controller, causes functions/operations specified in the flowchart and/or the block diagram to be implemented. The program code may be executed entirely on a machine, partly on a machine, partly on a machine as a stand-alone software package and partly on a remote machine, or entirely on a remote machine or a server.

In the context of the present disclosure, the machine readable medium may be a tangible medium which may contain or store a program for use by or in connection with an instruction execution system, apparatus, or device. The machine readable medium may be a machine readable signal medium or a machine readable storage medium. The machine readable medium may include, but is not limited to, an electronic, magnetic, optical, electromagnetic, infrared, or semiconductor system, apparatus, or device, or any suitable combination of the foregoing. More specific examples of the machine readable storage medium may include an electrical connection based on one or more wires, a portable computer disk, a hard disk, a random access memory (RAM), a read only memory (ROM), an erasable programmable read only memory (EPROM or flash memory), an optical fiber, a portable compact disc read only memory (CD-ROM), an optical storage device, a magnetic storage device, or any suitable combination of the foregoing.

To provide interaction with a user, the systems and technologies described here may be implemented on a computer having: a display device (for example, a cathode ray tube (CRT) or liquid crystal display (LCD) monitor) for displaying information to a user; and a keyboard and a pointing device (for example, a mouse or a trackball) by which a user may provide input for the computer. Other kinds of devices may also be used to provide interaction with a user; for example, feedback provided for a user may be any form of sensory feedback (for example, visual feedback, auditory feedback, or tactile feedback); and input from a user may be received in any form (including acoustic, speech or tactile input).

The systems and technologies described here may be implemented in a computing system (for example, as a data server) which includes a back-end component, or a computing system (for example, an application server) which includes a middleware component, or a computing system (for example, a user computer having a graphical user interface or a web browser through which a user may interact with an implementation of the systems and technologies described here) which includes a front-end component, or a computing system which includes any combination of such back-end, middleware, or front-end components. The components of the system may be interconnected through any form or medium of digital data communication (for example, a communication network). Examples of the communication network include: a local area network (LAN), a wide area network (WAN), the Internet and a blockchain network.

A computer system may include a client and a server. Generally, the client and the server are remote from each other and interact through the communication network. The relationship between the client and the server is generated by virtue of computer programs which run on respective computers and have a client-server relationship to each other. The server may be a cloud server, also called a cloud computing server or a cloud host, and is a host product in a cloud computing service system, so as to overcome the defects of high management difficulty and weak service expansibility in conventional physical host and virtual private server (VPS) service. The server may also be a server of a distributed system, or a server incorporating a blockchain.

The technology according to the present disclosure provides a more efficient ranking learning model, and the plurality of drugs corresponding to the same target protein may be ranked more efficiently and more accurately.

It should be understood that various forms of the flows shown above may be used and reordered, and steps may be added or deleted. For example, the steps described in the present disclosure may be executed in parallel, sequentially, or in different orders, which is not limited herein as long as the desired results of the technical solution disclosed in the present disclosure may be achieved.

The above-mentioned implementations are not intended to limit the scope of the present disclosure. It should be understood by those skilled in the art that various modifications, combinations, sub-combinations and substitutions may be made, depending on design requirements and other factors. Any modification, equivalent substitution and improvement made within the spirit and principle of the present disclosure all should be included in the extent of protection of the present disclosure.

What is claimed is:

1. A method for training a ranking learning model, comprising:
collecting a plurality of training samples, each of the plurality of training samples comprising information of a known training target protein, information of two training drugs, and a real difference between affinities of the two training drugs for the known training target protein; and
training the ranking learning model with the plurality of training samples, such that the ranking learning model learns a capability of predicting a magnitude relationship between the affinities of the two training drugs for the known training target protein in each of the plurality of training samples.

2. The method according to claim 1, wherein training the ranking learning model with the plurality of training samples, such that the ranking learning model learns the capability of predicting the magnitude relationship between the affinities of the two training drugs for the known training target protein in each of the plurality of training samples comprises:
inputting the information of the known training target protein and the information of the two training drugs in each of the plurality of training samples into the ranking learning model;
acquiring a predicted difference between the affinities of the two training drugs for the known training target protein output by the ranking learning model; and
adjusting parameters of the ranking learning model based on the predicted difference between the affinities and the real difference between the affinities, such that the ranking learning model learns the capability of predicting the magnitude relationship between the affinities of the two training drugs for the known training target protein in each of the plurality of training samples.

3. The method according to claim 2, wherein adjusting parameters of the ranking learning model based on the predicted difference between the affinities and the real difference between the affinities, such that the ranking learning model learns the capability of predicting the magnitude relationship between the affinities of the two training drugs for the known training target protein in each of the plurality of training samples comprises:
constructing a loss function based on the predicted difference between the affinities and the real difference between the affinities;
detecting whether the loss function converges; and
if the loss function does not converge, adjusting the parameters of the ranking learning model, such that the loss function tends to converge.

4. The method according to claim 1, wherein the collecting the plurality of training samples comprises:
collecting the plurality of training samples from a plurality of data sets.

5. The method according to claim 4, wherein the affinities of the training drugs for the known training target in different data sets are characterized by different indexes.

6. The method according to claim 1, wherein the real difference between affinities is a specific difference value or a value indicating a direction of the real difference between the affinities.

7. A drug ranking method, comprising:
acquiring information of an objective target and information of a plurality of candidate drugs; and
ranking the plurality of candidate drugs according to magnitude of their respective affinities for the objective target by a ranking model based on the information of the objective target and the information of the plurality of candidate drugs, wherein the ranking model shares parameters of a pre-trained ranking learning model, and the ranking learning mode is configured to learn a magnitude relationship between affinities of any two drugs for a same target protein.

8. An electronic device, comprising:
at least one processor; and
a memory connected with the at least one processor communicatively;
wherein the memory stores instructions executable by the at least one processor to cause the at least one processor to carry out a method for training a ranking learning model, which comprises:
collecting a plurality of training samples, each of the plurality of training samples comprising information of a known training target protein, information of two training drugs, and a real difference between affinities of the two training drugs for the known training target protein; and
training the ranking learning model with the plurality of training samples, such that the ranking learning model learns a capability of predicting a magnitude relationship between the affinities of the two training drugs for the known training target protein in each of the plurality of training samples.

9. The electronic device according to claim 8, wherein training the ranking learning model with the plurality of training samples, such that the ranking learning model learns the capability of predicting the magnitude relationship between the affinities of the two training drugs for the known training target protein in each of the plurality of training samples comprises:
inputting the information of the known training target protein and the information of the two training drugs in each of the plurality of training samples into the ranking learning model;
acquiring a predicted difference between the affinities of the two training drugs for the known training target protein output by the ranking learning model; and
adjusting parameters of the ranking learning model based on the predicted difference between the affinities and the real difference between the affinities, such that the ranking learning model learns the capability of predicting the magnitude relationship between the affinities of the two training drugs for the known training target protein in each of the plurality of training samples.

10. The electronic device according to claim 9, wherein adjusting parameters of the ranking learning model based on the predicted difference between the affinities and the real difference between the affinities, such that the ranking learning model learns the capability of predicting the magnitude relationship between the affinities of the two training drugs for the known training target protein in each of the plurality of training samples comprises:
constructing a loss function based on the predicted difference between the affinities and the real difference between the affinities;
detecting whether the loss function converges; and
if the loss function does not converge, adjusting the parameters of the ranking learning model, such that the loss function tends to converge.

11. The electronic device according to claim 8, wherein the collecting the plurality of training samples comprises:
collecting the plurality of training samples from a plurality of data sets.

12. The electronic device according to claim 11, wherein the affinities of the training drugs for the known training target in different data sets are characterized by different indexes.

13. The electronic device according to claim 8, wherein the real difference between affinities is a specific difference value or a value indicating a direction of the real difference between the affinities.

14. An electronic device, comprising:
at least one processor; and
a memory connected with the at least one processor communicatively;
wherein the memory stores instructions executable by the at least one processor to cause the at least one processor to carry out a drug ranking method, which comprises:
acquiring information of an objective target and information of a plurality of candidate drugs; and
ranking the plurality of candidate drugs according to magnitude of their respective affinities for the objective target by a ranking model based on the information of the objective target and the information of the plurality of candidate drugs, wherein the ranking model shares parameters of a pre-trained ranking learning model, and the ranking learning mode is configured to learn a magnitude relationship between affinities of any two drugs for a same target protein.

15. A non-transitory computer-readable storage medium comprising computer instructions, which, when executed by a computer, cause the computer to carry out a method for training a ranking learning model, which comprises:
collecting a plurality of training samples, each of the plurality of training samples comprising information of a known training target protein, information of two training drugs, and a real difference between affinities of the two training drugs for the known training target protein; and
training the ranking learning model with the plurality of training samples, such that the ranking learning model learns a capability of predicting a magnitude relationship between the affinities of the two training drugs for the known training target protein in each of the plurality of training samples.

16. The non-transitory computer-readable storage medium according to claim 15, wherein training the ranking learning model with the plurality of training samples, such that the ranking learning model learns the capability of predicting the magnitude relationship between the affinities of the two training drugs for the known training target protein in each of the plurality of training samples comprises:
inputting the information of the known training target protein and the information of the two training drugs in each of the plurality of training samples into the ranking learning model;
acquiring a predicted difference between the affinities of the two training drugs for the known training target protein output by the ranking learning model; and
adjusting parameters of the ranking learning model based on the predicted difference between the affinities and the real difference between the affinities, such that the ranking learning model learns the capability of predicting the magnitude relationship between the affinities of the two training drugs for the known training target protein in each of the plurality of training samples.

17. The non-transitory computer-readable storage medium according to claim 16, wherein adjusting parameters of the ranking learning model based on the predicted difference between the affinities and the real difference between the affinities, such that the ranking learning model learns the capability of predicting the magnitude relationship between the affinities of the two training drugs for the known training target protein in each of the plurality of training samples comprises:
constructing a loss function based on the predicted difference between the affinities and the real difference between the affinities;
detecting whether the loss function converges; and
if the loss function does not converge, adjusting the parameters of the ranking learning model, such that the loss function tends to converge.

18. The non-transitory computer-readable storage medium according to claim 15, wherein the collecting the plurality of training samples comprises:
collecting the plurality of training samples from a plurality of data sets.

19. The non-transitory computer-readable storage medium according to claim 18, wherein the affinities of the training drugs for the known training target in different data sets are characterized by different indexes.

20. A non-transitory computer-readable storage medium comprising computer instructions, which, when executed by a computer, cause the computer to carry out a drug ranking method, which comprises:
acquiring information of an objective target and information of a plurality of candidate drugs; and
ranking the plurality of candidate drugs according to magnitude of their respective affinities for the objective target by a ranking model based on the information of the objective target and the information of the plurality of candidate drugs, wherein the ranking model shares parameters of a pre-trained ranking learning model, and the ranking learning mode is configured to learn a magnitude relationship between affinities of any two drugs for a same target protein.
